(19) United States
(12) Patent Application Publication
(10) Pub. No.: US 2025/0221805 A1
(43) Pub. Date: Jul. 10, 2025

(54) ORAL CARE DEVICE, CONTROL METHOD THEREOF, AND CONTROL DEVICE THEREOF

(71) Applicant: GUANGZHOU STARS PULSE CO.,LTD., GUANGZHOU (CN)

(72) Inventors: YU ZOU, GUANGZHOU (CN); WENQIANG LIN, GUANGZHOU (CN)

(21) Appl. No.: 19/010,641

(22) Filed: Jan. 6, 2025

(30) Foreign Application Priority Data
Jan. 5, 2024 (CN) 202410025158.8
May 9, 2024 (CN) 202410572179.1

(51) Int. Cl. A61C 17/22; A46B 15/00

(52) U.S. Cl. CPC A61C 17/221; A46B 15/004

(57) ABSTRACT

An oral care device, a control method thereof, a control device thereof, and a storage medium are provided. The control method includes steps of acquiring at least one of oral care motion change information and oral care pressure change information of the oral care device; and controlling, according to at least one of the oral care motion change information and the oral care pressure change information, the oral care device to output at least one of a tone, a sound intensity, and a rhythm of a predetermined sound to generate a corresponding change.

### TECHNICAL FIELD

The present disclosure relates to a technical field of oral care, and in particular to an oral care device, a control method thereof, and a control device thereof.

### BACKGROUND

Electric toothbrushes become more and more popular. An electric toothbrush with music playback function is generally made by adding a music chip, a playback circuit, and a small speaker to basic structures of the electric toothbrush. Alternatively, in another electric toothbrush with music playback function, a motor thereof is connected to an audio amplifier circuit, and then the motor drives a brush head to vibrate according to an audio driving signal transmitted by the audio amplifier circuit, so the electric toothbrush is capable of playing music while brushing teeth.

However, if the electric toothbrush generates sound through vibrations of the motor, a tone, a sound intensity, or a rhythm of a generated sound is fixed, resulting in a poor interaction between the electric toothbrush and the user when the generated sound is emitted.

### SUMMARY

Embodiments of the present disclosure provide a control method of an oral care device, a control device of the control device, and an electronic device, which allow a user to feel sound output changes brought by changes in oral care operations while performing the oral care operations, making an oral care process like a music performance process, motivating the user to perform oral care, and cultivating his/her interest in oral care. Technical solutions are as follow.

In a first aspect, the present disclosure provides the control method of the oral care device. The control method is applied to the oral care device, and the control method includes steps of acquiring at least one of oral care motion change information and oral care pressure change information of the oral care device; and controlling, according to at least one of the oral care motion change information and the oral care pressure change information, the oral care device to output at least one of a tone, a sound intensity, and a rhythm of a predetermined sound to generate a corresponding change.

In a second aspect, the present device provides the control device of the oral care device. The control device is applied to the oral care device. The control devices includes an acquisition module and a first control module. The acquisition module is configured to acquire at least one of oral care motion change information and oral care pressure change information of the oral care device. The first control module is configured to control the oral care device to output at least one of a tone, a sound intensity, and a rhythm of a predetermined sound to generate a corresponding change according to at least one of the oral care motion change information and the oral care pressure change information.

In a third aspect, the present disclosure provides the oral care device. The oral care device includes a memory and a processor. The memory is connected to the processor. The memory is configured to store executable program codes. The processor runs programs corresponding to the executable program codes by reading the executable program codes stored in the memory. The processor runs the programs to execute the steps in the first aspect of the present disclosure.

### BRIEF DESCRIPTION OF DRAWINGS

In order to clearly describe technical solutions in the embodiments of the present disclosure, the following will briefly introduce the drawings that need to be used in the description of the embodiments or the prior art. Apparently, the drawings in the following description are merely some of the embodiments of the present disclosure, and those skilled in the art are able to obtain other drawings according to the drawings without contributing any inventive labor.

FIG. 1 is a structural schematic diagram of an oral care device according to one optional embodiment of the present disclosure.

FIG. 2 is a flow chart of a control method of the oral care device according to one optional embodiment of the present disclosure.

FIG. 3 is a schematic diagram of an implementation process of the control method of the oral care device according to one optional embodiment of the present disclosure.

FIG. 4 is another schematic diagram of the implementation process of the control method of the oral care device according to one optional embodiment of the present disclosure.

FIG. 5 is another schematic diagram of the implementation process of the control method of the oral care device according to one optional embodiment of the present disclosure.

FIG. 6 is another schematic diagram of the implementation process of the control method of the oral care device according to one optional embodiment of the present disclosure.

FIG. 7 is another flow chart of the control method of the oral care device according to one optional embodiment of the present disclosure.

FIG. 8 is a schematic diagram of a control device of the oral care device according to one optional embodiment of the present disclosure.

FIG. 9 is another structural schematic diagram of the oral care device according to one optional embodiment of the present disclosure.

### DETAILED DESCRIPTION

Technical solutions in the embodiments of the present disclosure will be clearly and completely described below in conjunction with the accompanying drawings in the embodiments of the present disclosure. Obviously, the described embodiments are only a part of the embodiments of the present disclosure, rather than all of the embodiments. Based on the embodiments of the present disclosure, all other embodiments obtained by those of ordinary skill in the art without creative work shall fall within the protection scope of the present disclosure.

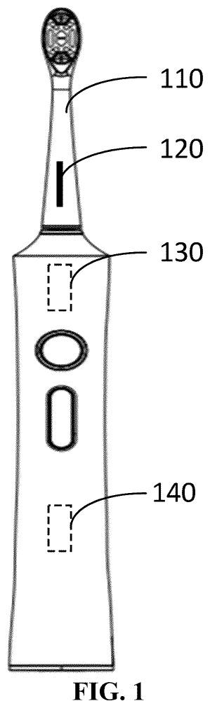

Please refer to FIG. 1, FIG. 1 is a structural schematic diagram of an oral care device according to one optional embodiment of the present disclosure. As shown in FIG. 1, the oral care device includes a detection element 110, a sound-generating piece 120, and a control unit 130. The oral care device is a device capable of cleaning an oral cavity, such as an electric toothbrush, which is not limited thereto. The embodiments below are all described by taking the oral care device as the electric toothbrush as an example.

The detection element 110 is configured to detect target oral care information of the oral care device. The detection element 110 includes, but is not limited to, a pressure detection element, an oral care motion detection element, etc. The pressure detection element may be, but is not limited to, detect a pressure on a care piece of the oral care device through any pressure sensing method such as a strain gauge, a Hall element magnetic induction, a capacitor, a resistor, an inductor, etc. The pressure detection element may be disposed at a motor shaft of the oral care device, the care piece (such as a brush head), a housing of a handle (such as a brush handle), a movement, a motor shell, or other position of the oral care device, which is not limited thereto. The detection element may, but is not limited to, detect the oral care motion information of the oral care device in a horizontal direction or a vertical direction through any sensor such as an acceleration sensor or a multi-axis sensor. For instance, the detection element may detect the number of times of oral care performed back and forth, and determine an oral care frequency corresponding to the oral care device according to the number of the times of the oral care performed back and forth in the horizontal direction or the vertical direction within a unit time (such as, but is not limited to 1 second, etc.). The oral care frequency includes, but is not limited to, an upper-lower oral care frequency (i.e., a upper-lower vertical brushing frequency in the vertical direction) and left-right oral care frequency (i.e., a left-right horizontal brushing frequency in the horizontal direction).

The sound-generating piece 120 may be, but is not limited to, a motor or a speaker. When the sound-generating piece 120 is the motor, the care piece of the oral care device generates a certain swing amplitude through vibrations of the motor, thereby breaking down toothpaste pasted on the care pieced into fine foam to achieve deep cleaning of the teeth. Further, the motor vibrates to drive the care piece to output a predetermined sound in a bone conduction manner. When the sound-generating piece is the speaker, the oral care device directly emit the predetermined sound through the speaker, which is not limited thereto.

The control unit 130 may be a microcontroller unit (MCU). The MCU is also known as a single-chip microcomputer or a single chip. The MCU appropriately reduces a frequency and specifications of a central process unit (CPU), and integrates peripheral interfaces such as memory, counter, USB, A/D conversion, universal asynchronous receiver/transmitter (UART), programmable logic controller (PLC), data memory access (DMA), and even liquid crystal display (LCD) drive circuits on a single chip to form a chip-level computer, which performs different combination controls for different application scenarios.

Specifically, the control unit 130 is connected to the sound-generating piece 120 and the detection element 110. After receiving at least one of oral care motion information and an oral care pressure of the oral care device detected by the detection element 110, the control unit 130 directly controls the sound-generating piece 120 to output at least one of a tone, a sound intensity, and a rhythm of the predetermined sound to generate the corresponding change according to at least one of oral care motion change information and oral care pressure change information.

Optionally, the oral care device shown in FIG. 1 may further include an indicator light, one or more buttons, the speaker, and a display screen, which is not limited thereto.

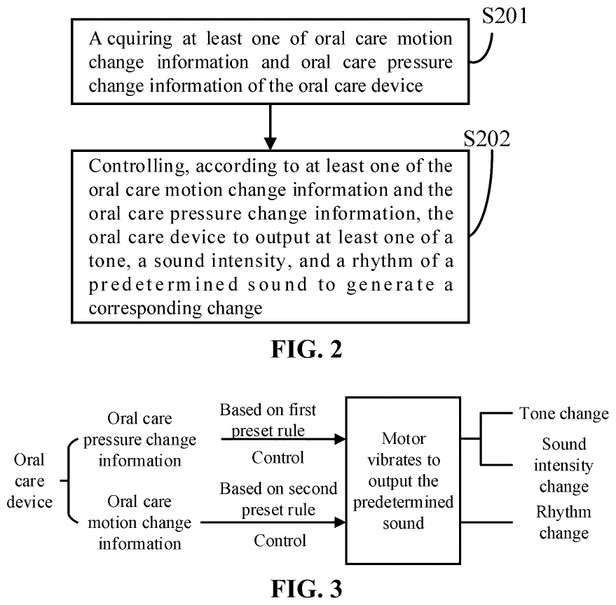

In conjunction with FIG. 1, a control method for the oral care device provided by one optional embodiment of the present disclosure is introduced by taking the control method applied to the electric toothbrush as an example. Please refer to FIG. 2, FIG. 2 is a flow chart of a control method of the oral care device according to one optional embodiment of the present disclosure. As shown in FIG. 2, the control method of the oral care device includes steps S201-S202.

The step S201 includes acquiring at least one of oral care motion change information and oral care pressure change information of the oral care device.

Specifically, when a user uses the oral cleaning device to clean the oral cavity, the oral cleaning device detects at least one of the oral care motion information and the oral care pressure of the oral care device through the detection element. Then, the oral cleaning device determine a corresponding oral care motion change information based on at least two oral care movement information detected continuously, and/or the oral care device determines a corresponding oral care pressure change information based on at least two oral care pressures detected continuously. The oral care pressures are obtained by detecting a pressure on the care piece through any pressure sensing method such as the strain gauge, the Hall element magnetic induction, the capacitor, the resistor, the inductor, etc., which is not limited thereto. The pressure sensing method is carried out by the pressure detection element, and the oral care motion information are obtained by detecting a motion of the oral care device back and forth for oral care (e.g., the number of times) through any sensor such as the acceleration sensor and the multi-axis sensor, which is not limited thereto.

The step S202 includes controlling, according to at least one of the oral care motion change information and the oral care pressure change information, the oral care device to output at least one of a tone, a sound intensity, and a rhythm of a predetermined sound to generate a corresponding change.

Specifically, the oral care device includes the sound-generating piece, and the he sound-generating piece is configured to control at least one of the tone, the sound intensity and rhythm of the predetermined sounds output by the sound-generating piece to change according to at least one of the oral care motion change information and the oral care pressure change information, which is not limited thereto.

Optionally, the sound-generating piece includes at least one of the speaker and the motor.

It is understood that during the oral care process of the oral care device, at least one of a current oral care mode, a current oral care duration, a current oral care pressure, and a current oral care time period of the oral care device is acquired, which is not limited thereto. When at least one of the current oral care mode, the current oral care duration, the current oral care pressure, and the current oral care time period of the oral care device changes, the predetermined sound output by the oral care device changes accordingly. The predetermined sound maybe predetermined music or predetermined voice, etc., which is not limited thereto. For instance, a corresponding oral care guidance voice is output according to the current oral care duration of the oral care device, so that oral care postures and oral care sequences of the user are guided by the corresponding oral care guidance voice. The predetermined music may also be output according to the current oral care mode of the oral care device, so that the user is able to listen to music while performing the oral care. The prompt voice may also be output according to the current oral care pressure, so that the user is prompted to perform oral care with appropriate oral care pressure. Different types of music are able to be output according to different oral care time periods. For example, when the user performs the oral care in the morning, the oral care device outputs relatively energetic and relaxing music to help the user refresh his/her mind and mood, relieve stress, and enhance energy and vitality. Alternatively, when the user performs the oral care at night, the oral care device outputs tranquil meditation music to help the user relax and guide him or her into meditation or deep relaxation.

In the embodiment of the present disclosure, at least one of the oral care motion change information and the oral care pressure change information of the oral care device is acquired, and the oral care device is controlled to output at least one of the tone, the sound intensity, and the rhythm of the predetermined sound to generate the corresponding change according to at least one of the oral care motion change information and the oral care pressure change information.

Therefore, the user performing the oral care is allowed to change a current oral care operation, and the oral care device then controls at least one of the tone the sound intensity, and the rhythm of the predetermined sound according to at least one of the oral care motion change information and the oral care pressure change information to change, which allow the user to feel sound output changes brought by changes in the oral care operations while performing the oral care operations, making the oral care process like a music performance process, motivating the user to perform the oral care, and cultivating his/her interest in oral care.

Optionally, the sound-generating piece may include, but is not limited to, the speaker. The step S202 of controlling, according to at least one of the oral care motion change information and the oral care pressure change information, the oral care device to output at least one of the tone, the sound intensity, and the rhythm of the predetermined sound to generate the corresponding change includes, but is not limited to, controlling, according to at least one of the oral care motion change information and the oral care pressure change information, the speaker in a predetermined manner to output at least one of the tone, the sound intensity, and the rhythm of the predetermined sound to generate the corresponding change. For instance, at least one of a driving voltage and a driving frequency of the speaker is changed according to at least one of the oral care motion change information and the oral care pressure change information, so that at least one of the tone, the sound intensity, and the rhythm of the predetermined sound emitted by the speaker changes.

Optionally, the oral care device includes one or more predetermined sounds stored therein. Before the step S202 of controlling, according to at least one of the oral care motion change information and the oral care pressure change information, the sound-generating piece to output at least one of the tone, the sound intensity, and the rhythm of the predetermined sound to generate the corresponding change, the predetermined sound to be played is acquired and a first driving signal is generated according to the predetermined sound to be played. The step S202 of controlling, according to at least one of the oral care motion change information and the oral care pressure change information, the sound-generating piece to output at least one of the tone, the sound intensity, and the rhythm of the predetermined sound to generate the corresponding change includes but is not limited to updating the first driving signal according to at least one of the oral care motion change information and the oral care pressure change information to obtain a second driving signal, and driving, according to the second driving signal, the sound-generating piece to output at least one of the tone, the sound intensity, and the rhythm of the predetermined sound to generate the corresponding change.

Furthermore, the sound-generating piece includes the speaker, and the oral care device further includes a power amplifier. The step of driving, according to the second driving signal, the sound-generating piece to output at least one of the tone, the sound intensity, and the rhythm of the predetermined sound to generate the corresponding change includes, but is not limited to, transmitting the second driving signal to the power amplifier to make the power amplifier to amplify the second driving signal, and driving the speaker to output at least one of the tone, the sound intensity, and the rhythm of the predetermined sound to generate the corresponding change according to an amplified second driving signal.

Optionally, the sound-generating piece includes the motor. The step S202 of controlling, according to at least one of the oral care motion change information and the oral care pressure change information, the sound-generating piece to output at least one of the tone, the sound intensity, and the rhythm of the predetermined sound to generate the corresponding change includes, but is not limited to, controlling, according to at least one of the oral care motion change information and the oral care pressure change information, the motor to vibrate to output at least one of the tone, the sound intensity, and the rhythm of the predetermined sound to generate the corresponding change.

In one optional embodiment, as shown in FIG. 3, the step of controlling, according to at least one of the oral care motion change information and the oral care pressure change information, the motor to vibrate to output at least one of the tone, the sound intensity, and the rhythm of the predetermined sound to generate the corresponding change includes steps of controlling, according to the oral care pressure change information, the motor to vibrate, based on a first predetermined rule, to output at least one of the tone and the sound intensity of the predetermined sound to generate the corresponding change (for example, but not limited to, adjusting a vibration frequency of the motor according to the oral care pressure change information to control the motor to vibrate to output the tone of the predetermined sound to generate the corresponding change; and/or, adjusting a vibration amplitude of the motor according to the oral care pressure change information to control the motor to vibrate to output the sound intensity of the predetermined sound to generate the corresponding change, etc.); and/or controlling, according to the oral care motion change information, the motor to vibrate, based on a second predetermined rule, to output the rhythm of the predetermined sound to generate the corresponding change (for example, but not limited to, determining change information of the oral care frequency of the oral care device according to the oral care motion change information, where the oral care frequency represents the number of times the oral care device performs the oral care back and forth in the vertical direction or the horizontal direction within a unit time (for example, but not limited to 1 second, etc.); then, the motor is controlled to vibrate to output the rhythm of the predetermined sound to generate the corresponding change according to the change information of the oral care frequency, so that the user is able to actively control the rhythm generated by the vibrations of the motor of the oral care device by manually adjusting the upper-lower oral care frequency and the left-right oral care frequency, thereby improving the interactivity and interest between the user and the oral care device during the oral care process.).

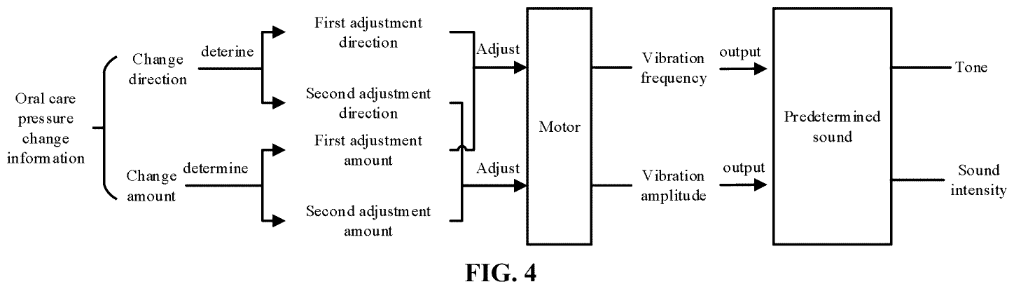

Furthermore, as shown in FIG. 4, the oral care pressure change information includes a change direction and a change amount of the oral care pressure on a care piece of the oral care device. An Implementation process of adjusting the vibration frequency of the motor according to the oral care pressure change information includes determining a first adjustment direction corresponding to the vibration frequency of the motor according to the change direction of the oral care pressure; determining a first adjustment amount corresponding to the vibration frequency of the motor according to the change amount of the oral care pressure; and adjusting the vibration frequency of the motor according to the first adjustment direction and the first adjustment amount. Therefore, the user is able to manually adjust his or her own oral care pressure to change the tone of the predetermined sound output by the oral care device, the oral care process is similar to the music performance process, motivating the user to perform the oral care and cultivating the interest of the user in oral care.

It is understood that the change direction of the oral care pressure may be the same as the first adjustment direction corresponding to the vibration frequency of the motor. For instance, when the oral care pressure increases, the vibration frequency of the motor is increased accordingly. Therefore, when the care piece of the oral care device is in contact with the tooth surface due to an increase in the oral care pressure, the vibration frequency of the motor is increased to drive the care piece to generate sufficient friction to achieve a better cleaning effect. Moreover, by increasing the vibration frequency of the motor, the tone of the predetermined sound output by the vibrations of the motor increases accordingly, so that the user is able to adjust tone of the predetermined sound output by the oral care device to a desired tone by manually adjusting the oral care pressure.

It is understood that the change direction of the oral care pressure may be opposite to the first adjustment direction corresponding to the vibration frequency of the motor. For instance, the greater the oral care pressure, the lower the vibration frequency of the motor. Therefore, when the oral care pressure increases, the vibration frequency of the motor is reduced to reduce wear and irritation to the teeth and gums, so that the oral care device performs more gentle oral care and improve the comfort of use. Moreover, by reducing the vibration frequency of the motor, the tone of the predetermined sound output by the vibrations of the motor reduces accordingly, thereby improving the interactivity and fun of the user with the oral care device during the oral care process. The smaller the oral care pressure, the higher the vibration frequency of the motor. Therefore, when the oral care pressure reduces, a higher oral cleaning ability is provided through a larger vibration frequency of the motor. Further, as the vibration frequency of the motor increases, the tone of the predetermined sound output by the vibrations of the motor increase, thereby improving the interactivity and fun of the user with the oral care device during the oral care process.

It is understood that a correspondence between the change amount of the oral care pressure and the adjustment amount corresponding to the vibration frequency of the motor is predetermined, and then the first adjustment amount corresponding to the vibration frequency of the motor is determined based on the correspondence and a current change amount of the oral care pressure. In order to improve the interactivity between the user and the oral care device while achieving a better oral care effect, when the change direction of the oral care pressure changes, the correspondence between the change amount of the oral care pressure and the adjustment amount corresponding to the vibration frequency of the motor changes accordingly, and/or when the pressure range of the oral care pressure changes, the correspondence between the change amount of the oral care pressure and the adjustment amount corresponding to the vibration frequency of the motor changes accordingly, etc., which is not limited thereto.

Furthermore, as shown in FIG. 4, the oral care pressure change information includes the change direction and the change amount of the oral care pressure on a care piece of the oral care device. An implementation process of adjusting the vibration amplitude of the motor according to the oral care pressure change information includes determining a second adjustment direction corresponding to the vibration amplitude of the motor according to the change direction of the oral care pressure; determining a second adjustment amount corresponding to the vibration amplitude of the motor according to the change amount of the oral care pressure; and adjusting the vibration amplitude of the motor according to the second adjustment direction and the second adjustment amount. Therefore, the user is allowed to manually adjust the own oral care pressure to adjust the sound intensity of the predetermined sound output by the oral care device, thereby improving the interactivity and fun between the user and the oral care device during the oral care process.

It is understood that the change direction of the oral care pressure may be the same as the second adjustment direction corresponding to the vibration amplitude of the motor. For instance, when the oral care pressure increases, the vibration amplitude of the motor is increased accordingly. Therefore, when the care piece of the oral care device is closely in contact with the tooth surface due to the increase in the oral care pressure, the vibration amplitude of the motor is increased to remove tartar and plaque from the tooth surfaces, so as to achieve a better cleaning effect. Moreover, by increasing the vibration amplitude of the motor, the sound intensity of the predetermined sound output by the vibrations of the motor increases accordingly, so that the user is able to adjust sound intensity of the predetermined sound output by the oral care device to a desired sound intensity by manually adjusting the oral care pressure.

It is understood that the change direction of the oral care pressure may be opposite to the second adjustment direction corresponding to the vibration amplitude of the motor. For instance, the greater the oral care pressure, the lower the vibration amplitude of the motor. Therefore, when the oral care pressure increases, the vibration amplitude of the motor is reduced to reduce wear and irritation to the teeth and gums, so that the oral care device performs more gentle oral care and improve the comfort of use. Moreover, by reducing the vibration amplitude of the motor, the sound intensity of the predetermined sound output by the vibrations of the motor reduces accordingly, thereby improving the interactivity and fun of the user with the oral care device during the oral care process. The smaller the oral care pressure, the higher the vibration amplitude of the motor. Therefore, when the oral care pressure reduces, a higher oral cleaning ability is provided through a larger vibration amplitude of the motor. Further, as the vibration amplitude of the motor increases, the sound intensity of the predetermined sound output by the vibrations of the motor increase, thereby improving the interactivity and fun of the user with the oral care device during the oral care process.

It is understood that a correspondence between the change amount of the oral care pressure and the adjustment amount corresponding to the vibration amplitude of the motor is predetermined, and then the second adjustment amount corresponding to the vibration amplitude of the motor is determined based on the correspondence and the current change amount of the oral care pressure. In order to improve the interactivity between the user and the oral care device while achieving a better oral care effect, when the change direction of the oral care pressure changes, the correspondence between the change amount of the oral care pressure and the adjustment amount corresponding to the vibration amplitude of the motor changes accordingly, and/or when the pressure range of the oral care pressure changes, the correspondence between the change amount of the oral care pressure and the adjustment amount corresponding to the vibration amplitude of the motor changes accordingly, etc., which is not limited thereto.

It is understood that when the oral care pressure of the oral care device is detected to be changed, the tone of the predetermined sound output by the vibrations of the motor may be adjusted only by adjusting the vibration frequency of the motor, or the sound intensity of the predetermined sound output by the vibrations of the motor may be adjusted only by adjusting the vibration amplitude of the motor, or the tone and the sound intensity of the predetermined sound output by the vibrations of the motor may be adjusted simultaneously by adjusting the vibration frequency and the vibration amplitude of the motor, etc., which is not limited thereto.

Furthermore, when the oral care pressure changes, in order to avoid relatively larger wear and irritation to the teeth and gums by the care piece of the oral care device when the vibration frequency and the vibration amplitude of the motor of the oral care device increase suddenly at the same time, and in order to avoid significant decrease in the cleaning effect of the care piece of the oral care device when the vibration frequency and the vibration amplitude of the motor of the oral care device reduce suddenly at the same time, when the change direction of the oral care pressure is kept unchanged, the first adjustment direction is opposite to the second adjustment direction. That is, when the vibration frequency and the vibration amplitude of the motor are adjusted simultaneously according to the oral care pressure change information, one of the vibration frequency and the vibration amplitude of the motor is increased, and the other one thereof is reduced, thereby improving control stability of the oral care device and the stability of the oral care effect, and improving the comfort of using the oral care device.

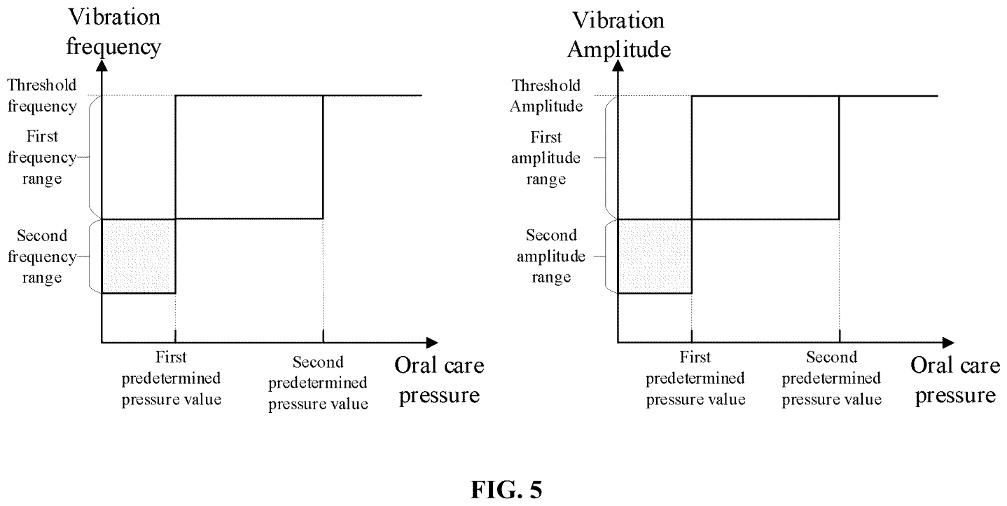

Optionally, as shown in FIG. 5, when the oral care pressure of the oral care device is not less than a first predetermined pressure value and is not greater than a second predetermined pressure value, it is determined that the oral care pressure of the oral care device is within a normal oral care pressure range, that is, the user is currently performing normal oral care operations. At this time, the motor is controlled to vibrate within a first frequency range and a first amplitude range according to the oral care pressure change information, so as to control the motor to vibrate to output at least one of the tone and the sound intensity of the predetermined sound to generate the corresponding change. The second predetermined pressure value is greater than the first predetermined pressure value. That is, no matter whether the vibration frequency of the motor or the vibration amplitude of the motor is adjusted according to the oral care pressure, the vibration frequency and vibration amplitude of the motor change within a certain range. For instance, when the oral care pressure changes to be greater than the second predetermined pressure value, even if the oral care pressure is still increasing, the vibration frequency and vibration amplitude of the motor no longer change. Instead, the motor is controlled to vibrate at a boundary frequency and a boundary amplitude corresponding to the second predetermined pressure value within the first frequency range and the first amplitude range. Thus, the vibration frequency and the vibration amplitude of the oral care device are controlled to change only within the certain range according to the oral care pressure change information within the normal oral care pressure range, thereby avoiding excessive changes in the vibration frequency and the vibration amplitude of the motor that affect the oral care effect of the oral care device, while ensuring the continuity and clarity of the predetermined sound output by the vibrations of the motor of the oral care device.

It is understood that the first predetermined pressure value and the second predetermined pressure value may be determined according to actual oral care conditions, which are not limited thereto.

As shown in FIG. 5, when the oral care pressure is less than the first predetermined pressure value, it is determined that the care piece does not effectively contact the teeth, and the motor is controlled to vibrate in a second frequency range and a second amplitude range, or the motor is controlled to vibrate to output a pressure-too-small prompt sound. A minimum value of the first frequency range is not less than a maximum value of the second frequency range, and a minimum value of the first amplitude range is not less than a maximum value of the second amplitude range.

Therefore, by controlling the motor to vibrate within the second frequency range and the second amplitude range, it is possible to avoid the problem that the oral care device motor still vibrates at a large vibration frequency and a large vibration amplitude and emits the predetermined sound when the care piece is not in effective contact with the teeth, which consumes a lot of energy and causes the energy of the oral care device to be wasted ineffectively. Therefore, an effectiveness of the oral care device in outputting the predetermined sound is improved. Alternatively, the motor vibrates to output pressure-too-small prompt sound to prompt the user that the current oral care pressure is too low, thereby assisting the user to adjust invalid oral care operations in time to ensure the oral care effect.

Furthermore, when the oral care pressure is less than the first predetermined pressure value, a process of controlling the motor to vibrate in the second frequency range and the second amplitude range includes: when the oral care pressure changes from being not less the first predetermined pressure value to being less than the first predetermined pressure value, and the duration of the oral care pressure being less than the first predetermined pressure value is greater than the predetermined duration (for example, but not limited to, 3 seconds, 5 seconds, etc.), the motor is controlled to adjust the vibration frequency to the second frequency range and reduce the vibration amplitude to the second amplitude range. That is, when the oral care pressure is reduced to the first predetermined pressure value, the vibration frequency and the vibration amplitude of the motor do not reduce immediately, so as to avoid sudden wrong operations of the user. For example, the user may suddenly lift the oral care device or perform the oral care to change a brushing area during the oral care process, and as a result, the oral care pressure is temporarily less than the first predetermined pressure value, resulting in frequent and large changes in the vibration frequency and the vibration amplitude of the motor, which affect the continuity of the predetermined sound of the oral care device. Thus, the control stability of the oral care device and the continuity of the predetermined sound emitted are improved.

It is understood that when the oral care pressure is less than the first predetermined pressure value, the oral care device may control the motor to vibrate to output the predetermined sound, or he oral care device may control the motor to vibrate only at a small vibration frequency and a small vibration amplitude without outputting the predetermined sound, which is not limited thereto.

Optionally, as shown in FIG. 5, when the oral care pressure is greater than the second predetermined pressure value, the motor is controlled to vibrate at a threshold frequency and a threshold amplitude. That is, when the oral care pressure is greater than the second predetermined pressure value, the vibration frequency and the vibration amplitude of the motor no longer change with the change of the oral care pressure, which not only reminds the user that the current oral care pressure is too high, but also avoids problems that the vibration frequency and the vibration amplitude of the motor are too high to damage the teeth and gums of the use and the vibration frequency and the vibration amplitude of the motor are too small to affect the oral care effect.

It is understood that the threshold frequency is the boundary frequency within the first frequency range, such as but not limited to the maximum frequency (shown in the left mage of FIG. 5) or the minimum frequency within the first frequency range, and the threshold amplitude is the boundary amplitude within the first amplitude range, such as but not limited to the maximum amplitude (shown in the right image of FIG. 5) or the minimum amplitude within the first amplitude range, etc., which is not limited thereto.

Optionally, when the oral care pressure is greater than the second predetermined pressure value, the motor is controlled to vibrate to output a pressure-excessive prompt sound, thereby promptly reminding the user that the current oral care pressure is too high, and preventing the excessive oral care pressure from damaging the teeth and gums of the user and affecting the oral care effect.

Optionally, when the oral care device is turned on, the motor is controlled to vibrate (i.e., micro-vibrate) in the second frequency range and second amplitude range that are relatively small, thereby avoiding a problem of liquid splashing on the oral care device due to excessive vibration frequency and excessive vibration amplitude of the motor when the oral care device is turned on but is not placed in the oral cavity of the user. Further, the user is reminded by the micro-vibration that the oral care device is currently started. After the oral care device is turned on, when it is detected that the oral care pressure is greater than the first predetermined pressure value, it is determined that the care piece of the oral care device is currently effectively in contact with the teeth of the user, and the motor is controlled to vibrate in the first frequency range and the first amplitude range according to the oral care pressure change information, so that the motor is controlled to vibrate to output at least one of the tone and the sound intensity to change, thereby ensuring the oral care effect of the user while improving the interactivity and fun between the user and the oral care device during the oral care process.

It is understood that when the oral care frequency or the range of the oral care frequency changes, at least one of the vibration frequency, the vibration amplitude, and a rotation speed of the motor changes. That is, the rhythm of the predetermined sound output by the oral care device motor through the vibrations changes.

Optionally, an implementation process of controlling the motor to vibrate to output the rhythm of the predetermined sound to generate the corresponding change according to the change information of the oral care frequency includes: when the oral care frequency increases, the rhythm of the predetermined sound output by the vibrations of the motor speeds up, and when the oral care frequency reduces, the rhythm of the predetermined sound output by the vibrations of the motor slows down, thereby synchronizing the rhythm of the present sound generated by the motor with the change of the oral care frequency, improving the convenience of adjusting the rhythm of predetermined sound generated by the motor during the oral care process, and improving the interactivity and fun between the user and the oral care device.

Optionally, the implementation process of controlling the motor to vibrate to output the rhythm of the predetermined sound to generate the corresponding change according to the change information of the oral care frequency includes: when the oral care frequency is not less than a first predetermined frequency value and not greater than a second predetermined frequency value, it is determined that the user is currently using the oral care device to perform the oral care operations, and the motor is controlled to vibrate and output the rhythm of the predetermined sound to generate the corresponding change according to the change information of the oral care frequency. The second predetermined frequency value is greater than the first predetermined frequency value, so that the oral care process is similar to the music performance process, motivating the user to perform the oral care and cultivating the interest of the user in oral care.

Optionally, after the oral care device is turned on, when the oral care frequency is less than the first predetermined frequency value, it is determined that the user does not use the oral care device to perform oral care operations currently, and the motor is controlled to vibrate to output the predetermined sound at a first rhythm. When the oral care frequency is greater than the second predetermined frequency value, it is determined that the current oral care frequency is too fast, and the motor is controlled to vibrate to output the predetermined sound at a second rhythm to prompt the user that the current oral care frequency is too fast. The first rhythm is less than the second rhythm, so that the predetermined sound with obvious rhythm difference is output to respectively prompt the user that the current oral care device has been turned on with the predetermined sound of the smaller first rhythm, and prompt the user that the current oral care frequency is too fast with the predetermined sound of the larger second rhythm, so as to avoid the user from performing invalid or inefficient oral care operations all the time. Meanwhile, when the oral care frequency is too small (less than the first predetermined frequency value) or too high (greater than the second predetermined frequency value), the speed of the rhythm of the predetermined sound output by the vibrations of the motor through does not change with the change of the oral care frequency, which ensures the continuity and clarity of the predetermined sound emitted by the oral care device to a certain extent.

Optionally, an implementation process of controlling the motor to vibrate to output the rhythm of the predetermined sound to generate the corresponding change according to the change information of the oral care frequency includes: after the oral care device is turned on and detects that the oral care pressure changes from 0 to greater than the first predetermined pressure value, the motor is controlled to vibrate to output the rhythm of the predetermined sound to generate the corresponding change according to the change information of the oral care frequency, thereby avoiding a situation where the care piece does not effectively contact the teeth of the use after the oral care device is turned on and the user shakes the oral care device before starting oral care, causing the motor to ineffectively change the rhythm according to the oral care frequency detected by the shaking. Therefore, effectiveness and intelligence of the control of the oral care device are improved.

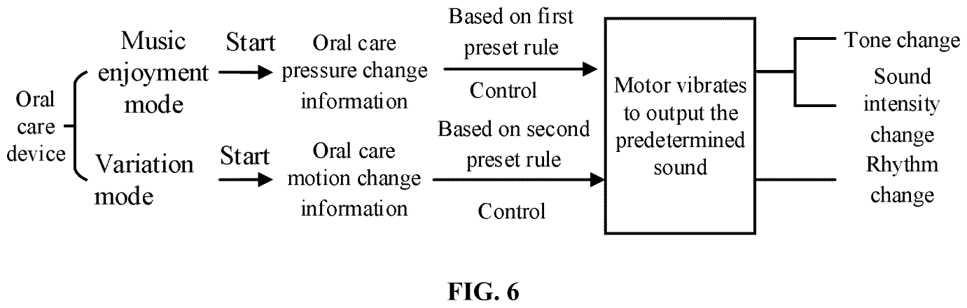

In some optional embodiments, as shown in FIG. 6, the oral care device has a pure music enjoyment mode and a variation mode. The implementation process of controlling, according to at least one of the oral care motion change information and the oral care pressure change information, the motor to output at least one of the tone, the sound intensity, and the rhythm of the predetermined sound to generate the corresponding change includes: when the pure music enjoyment mode of the oral care device is turned on, the motor is controlled to vibrate to output at least one of the tone and the sound intensity of the predetermined sound to generate the corresponding change based on the first predetermined rule according to the oral care pressure change information, so that the user is able to enjoy pure music during the oral care process and avoids the noise caused by the vibrations of the motor during the oral care process; and when the variation mode of the oral care device is turned on, the motor is controlled to vibrate to output the rhythm of the predetermined sound to generate the corresponding change based on the second predetermined rule according to the oral care motion change information, so that the user is able to control changes in melody of the predetermined sound output by the oral care device and experiences the fun of a conductor, and make the predetermined sound (such as music) output by the oral care device flow, giving the predetermined sound a different soul, thereby improving the interactivity and fun between the oral care device and the user.

Optionally, after the oral care device is turned on, the oral care device may, but is not limited to, obtain target identity information of the user who is using the oral care device for oral care. The oral care device may, but is not limited to, control the motor to vibrate to output at least one of the tone, the sound intensity, and the rhythm of the predetermined sound with a target timbre corresponding to the target identity information according to at least one of the oral care pressure and the oral care frequency of the oral care device. That is, the timbre of the predetermined sound output by the vibrations of the motor changes when the target identity information changes or attributes (for example, male users, female users, young users, elderly users, etc.) corresponding to the target identity information change, thereby improving the interactivity and fun between the user and the oral care device while improving the pertinence and flexibility of the control of the oral care device.

In some optional embodiments, when the oral care pressure of the oral care device is not less than the first predetermined pressure value and is not greater than the second predetermined pressure value, the motor is controlled to vibrate at a target vibration frequency and a target vibration amplitude to output the predetermined sound at a target tone and a target sound intensity. When the oral care pressure is less than the first predetermined pressure value or greater than the second predetermined pressure value, at least one of the tone, the sound intensity and the rhythm of the predetermined sound output by the vibrations of the motor is controlled to change. When the oral care pressure is less than the first predetermined pressure value or greater than the second predetermined pressure value, the tone of the predetermined sound output by the vibrations of the motor is less than the target tone, and/or the sound intensity of the predetermined sound output by the vibrations of the motor is less than the target sound intensity, and/or the rhythm of the predetermined sound output by the vibrations of the motor is less than a corresponding rhythm when the oral care pressure of the oral care device is not less than the first predetermined pressure value and not greater than the second predetermined pressure value, thereby allowing the predetermined sound output by the oral care device to generate relatively obvious changes within different pressure ranges. Further, when a current oral care pressure range of the oral care device changes, the user is intuitively and obviously reminded.

In some optional embodiments, after receiving a sound intensity change instruction input by the user or transmitted from the electronic device communicated with the oral care device, the oral care device responses to the sound intensity change instruction and controls the driving voltage corresponding to the sound-generating piece to change, so that the sound-generating piece outputs the sound intensity of the predetermined sound to generate the corresponding change. For example, but not limited to, when the sound intensity change instruction is a sound intensity reduction instruction, the driving voltage corresponding to the sound-generating piece is controlled to reduce, thereby reducing the sound intensity of the predetermined sound output by the sound-generating piece, and when the sound intensity change instruction is a sound intensity increase instruction, the driving voltage corresponding to the sound-generating piece is controlled to increase, thereby increasing the sound intensity of the predetermined sound output by the sound-generating piece.

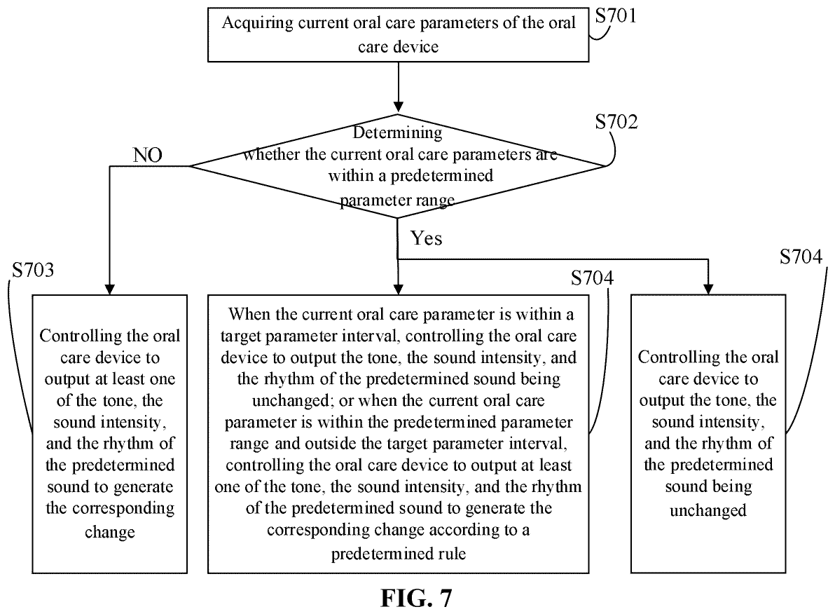

Please refer to FIG. 7, FIG. 7 is another flow chart of the control method of the oral care device according to one optional embodiment of the present disclosure. As shown in FIG. 7, the control method of the oral care device includes steps S701-S704.

The step S701 includes acquiring current oral care parameters of the oral care device.

Specifically, when the oral care device performs the oral care, the oral care device may, but is not limited to, acquire the current oral care parameters of the oral care device through the detection element. The current oral care parameters include, but not limited to, a current oral care progress, a current dental plaque residual amount, the current oral care pressure, the current oral care duration, a current oral care coverage rate, etc.

The step S702 includes determining whether the current oral care parameters are within a predetermined parameter range.

Specifically, after acquiring the current oral care parameters, the oral care device first determines whether the current oral care parameters are within the predetermined parameter range. The predetermined parameter range may include, but is not limited to, a predetermined oral care progress range corresponding to the current oral care progress, a predetermined dental plaque residual amount range corresponding to the current dental plaque residual amount, a predetermined oral care pressure range corresponding to the current oral care pressure, a predetermined oral care duration range corresponding to the current oral care duration, a predetermined oral care coverage rate range corresponding to the current oral care coverage rate, etc.

The step S703 includes when the current oral care parameters are outside the predetermined parameter range, controlling the oral care device to output at least one of the tone, the sound intensity, and the rhythm of the predetermined sound to generate the corresponding change.

Specifically, when the current oral care parameters are not within the predetermined parameter range, the sound-generating piece is controlled to output at least one of the tone, the sound intensity, and the rhythm of the predetermined sound to generate the corresponding change, thereby prompting the user that the current oral care parameters exceed the predetermined parameter range and it is necessary to adjust the oral care operations or the oral care device must enter a new oral care stage.

Optionally, after step S703 of when the current oral care parameters are outside the predetermined parameter range, controlling the oral care device to output at least one of the tone, the sound intensity, and the rhythm of the predetermined sound to generate the corresponding change, the control method of the oral care device includes, but is not limited to, the step S704. The step S704 includes: when the current oral care parameters are within the predetermined parameter range, controlling the oral care device to output the tone, the sound intensity, and the rhythm of the predetermined sound being unchanged; or when the current oral care parameter is within a target parameter interval, controlling the oral care device to output the tone, the sound intensity, and the rhythm of the predetermined sound being unchanged; or when the current oral care parameter is within the predetermined parameter range and outside the target parameter interval, controlling the oral care device to output at least one of the tone, the sound intensity, and the rhythm of the predetermined sound to generate the corresponding change according to a predetermined rule.

Specifically, when the current oral care parameters are within the predetermined parameter range, the tone, the sound intensity, and the rhythm of the predetermined sound output by the sound-generating piece are controlled to remain unchanged, thereby reminding the user that the current oral care parameters are within the predetermined parameter range. Alternatively, when the current oral care parameters are within the target parameter interval that is within the predetermined parameter range, the tone, the sound intensity, and the rhythm of the predetermined sound output by the sound-generating piece are controlled to remain unchanged. When the current oral care parameters are within the predetermined parameter range and outside the target parameter interval, at least one of the tone, the sound intensity, and the rhythm of the predetermined sound output by the sound-generating piece is controlled to change according to the predetermined rule. The target parameter interval may be, but is not limited to, a target pressure interval that ensures the oral care effect, a target oral care duration interval, a target oral care coverage interval, or a target oral care progress, when the oral care is not up to standard, thereby prompting the user whether the current oral care meets the standard by changing at least one of the tone, the sound intensity, and the rhythm according to the predetermined rule, so as to guide the user to achieve more efficient oral care and better oral care effect.

Optionally, the current oral care parameters include the current oral care pressure, and the predetermined parameter range includes a predetermined pressure range; and/or the predetermined rule includes that at least one of the tone and the sound intensity reduces first and then increases, or at least one of the tone and the sound intensity increases first and then reduces, or at least one of the tone and the sound intensity increases at a predetermined amplitude, or at least one of the tone and the sound intensity decreases with a predetermined amplitude, or at least one of the tone and the sound intensity repeatedly changes within a predetermined range; and/or the rhythm speeds up first and then slows down, or the rhythm slows down first then speeds up, or the rhythm slows down at the predetermined amplitude, or the rhythm speeds up at the predetermined amplitude, or the rhythm repeatedly changes within the predetermined range.

In some optional embodiments, the oral care device is communicated with the electronic device, and the electronic device includes a loudspeaker. After acquiring the current oral care parameters of the oral care device, the control method further include, but is not limited to, transmitting the current oral care parameters to the electronic device, and when the current oral care parameters are outside the predetermined parameter range, controlling, by the electronic device, the loudspeaker to output at least one of the tone, the sound intensity, and the rhythm of the predetermined sound.

Optionally, the electronic device may include but is not limited to a central console, the loudspeaker a terminal, etc.

Optionally, the current oral care parameters of the oral care device are acquired first, and then according to the change information of the parameter range of the current oral care parameters, the sound-generating piece is controlled to output at least one of the tone, the sound intensity, and the rhythm of the predetermined sound to generate the corresponding change. Specifically, when the parameter range of the current oral care parameters changes, at least one of the tone, the sound intensity, and the rhythm of the predetermined sound output by the sound-generating piece changes accordingly. For example but not limited to, the greater the pressure range of the current oral care pressure, the greater the sound intensity of the predetermined sound output by the sound-generating piece, and/or the lower the tone of the predetermined sound output by the sound-generating piece; the higher the frequency range of the current oral care frequency, the faster the rhythm of the predetermined sound output by the sound-generating piece, etc.

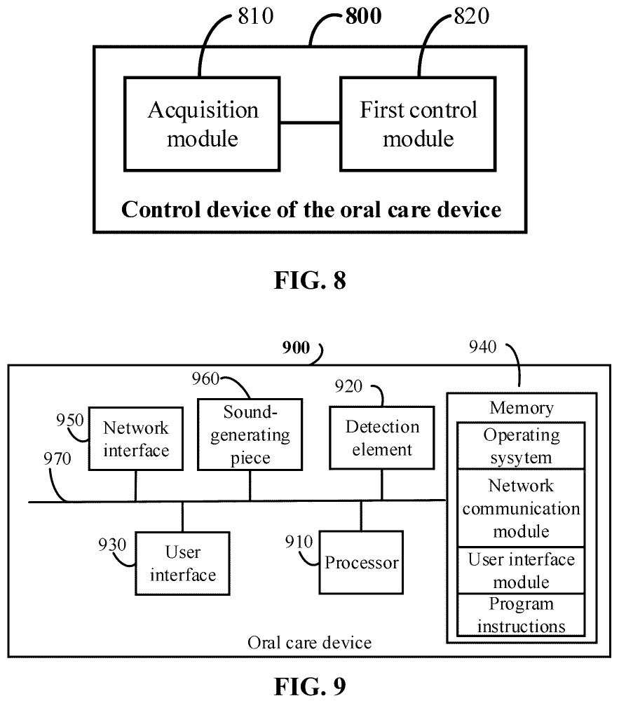

Please refer to FIG. 8, FIG. 8 is a schematic diagram of a control device of the oral care device according to one optional embodiment of the present disclosure. The control device of the oral care device is applied to the oral care device. As shown in FIG. 8, the control device 800 of the oral care device includes an acquisition module 810 and a first control module 820. The acquisition module 810 is configured to acquire at least one of oral care motion change information and oral care pressure change information of the oral care device. The first control module 820 is configured to control the oral care device to output at least one of a tone, a sound intensity, and a rhythm of a predetermined sound to generate a corresponding change according to at least one of the oral care motion change information and the oral care pressure change information.

In one optional embodiment, the oral care device includes a sound-generating piece. The first control module 820 is further configured to control the sound-generating piece to output at least one of the tone, the sound intensity, and the rhythm of the predetermined sound to generate the corresponding change according to at least one of the oral care motion change information and the oral care pressure change information.

In one optional embodiment, the sound-generating piece includes at least one of a speaker and a motor.

In one optional embodiment, the first control module 820 is specifically configured to control the motor to vibrate to output at least one of the tone, the sound intensity, and the rhythm of the predetermined sound to generate the corresponding change according to at least one of the oral care motion change information and the oral care pressure change information.

In one optional embodiment, the first control module 820 is specifically configured to control the speaker in a predetermined manner to output at least one of the tone, the sound intensity, and the rhythm of the predetermined sound to generate the corresponding change according to at least one of the oral care motion change information and the oral care pressure change information.

In one optional embodiment, before the step of controlling, according to at least one of the oral care motion change information and the oral care pressure change information, the sound-generating piece to output at least one of the tone, the sound intensity, and the rhythm of the predetermined sound to generate the corresponding change, the control device 800 of the oral care device further includes a sound acquisition module and a generation module. The sound acquisition module is configured to acquire the predetermined sound. The generation module is configured to generate a first driving signal according to the predetermined sound. The first control module 820 is specifically configured to update the first driving signal according to at least one of the oral care motion change information and the oral care pressure change information to obtain a second driving signal; and the first control module 820 is specifically configured to drive the sound-generating piece to output at least one of the tone, the sound intensity, and the rhythm of the predetermined sound to generate the corresponding change according to the second driving signal.

In one optional embodiment, the sound-generating piece includes the speaker, and the oral care device further includes a power amplifier. When the first control module 820 drives the sound-generating piece to output at least one of the tone, the sound intensity, and the rhythm of the predetermined sound to generate the corresponding change according to the second driving signal, the first control module 820 is specifically configured to transmit the second driving signal to the power amplifier to make the power amplifier to amplify the second driving signal, and drive the speaker to output at least one of the tone, the sound intensity, and the rhythm of the predetermined sound to generate the corresponding change according to an amplified second driving signal.

In one optional embodiment, the control device 800 of the oral care device further includes a first receiving module and a second control module. The first receiving module is configured to receive a sound intensity change instruction. The second control module is configured to control a driving voltage corresponding to the sound-generating piece to change in response to the sound intensity change instruction, so that the sound-generating piece outputs the sound intensity of the predetermined sound to generate the corresponding change.

In one optional embodiment, the control device 800 of the oral care device further includes a parameter acquisition module, a judgment module, and a third control module. The parameter acquisition module is configured to acquire current oral care parameters of the oral care device. The judgment module is configured to determine whether the current oral care parameters are within a predetermined parameter range. The third control module is configured to control the oral care device to output at least one of the tone, the sound intensity, and the rhythm of the predetermined sound to generate the corresponding change when the current oral care parameters are outside the predetermined parameter range.

In one optional embodiment, the control device 800 of the oral care device also includes a fourth control module. The fourth control module is configured to control the oral care device to output the tone, the sound intensity, and the rhythm of the predetermined sound being unchanged when the current oral care parameters are within the predetermined parameter range. Alternatively, the fourth control module is configured to control the oral care device to output the tone, the sound intensity, and the rhythm of the predetermined sound being unchanged when the current oral care parameters are within a target parameter interval. The fourth control module is further configured to control the oral care device to output at least one of the tone, the sound intensity, and the rhythm of the predetermined sound to generate the corresponding change according to a predetermined rule when the current oral care parameters are within the predetermined parameter range and outside the target parameter interval.

In one optional embodiment, the current oral care parameters include a current oral care pressure, and the predetermined parameter range includes a predetermined pressure range; and/or the predetermined rule includes that at least one of the tone and the sound intensity reduces first and then increases, at least one of the tone and the sound intensity increases first and then reduces, at least one of the tone and the sound intensity increases at a predetermined amplitude, at least one of the tone and the sound intensity decreases with a predetermined amplitude, or at least one of the tone and the sound intensity repeatedly changes within a predetermined range; and/or the rhythm speeds up first and then slows down, or the rhythm slows down first then speeds up, or the rhythm slows down at the predetermined amplitude, or the rhythm speeds up at the predetermined amplitude, or the rhythm repeatedly changes within the predetermined range.

In one optional embodiment, the oral care device is communicated with an electronic device, and the electronic device includes a loudspeaker. The control device 800 of the oral care device includes a parameter acquisition module and a sending module. The parameter acquisition module is configured to acquire current oral care parameters of the oral care device. The sending module is configured to transmit the current oral care parameters to the electronic device. When the current oral care parameters are outside the predetermined parameter rang, the sending module is configured to control the loudspeaker to output at least one of the tone, the sound intensity, and the rhythm of the predetermined sound.

In one optional embodiment, the electronic device includes at least one of a central console, the loudspeaker, and a terminal.

In one optional embodiment, the control device 800 of the oral care device further includes the parameter acquisition module and a control module. The parameter acquisition module is configured to acquire the current oral care parameters. The control module is configured to control the sound-generating piece to output at least one of the tone, the sound intensity, and the rhythm of the predetermined sound to generate the corresponding change according to the change information of the parameter range of the current oral care parameters, Specifically, when the parameter range of the current oral care parameters changes, at least one of the tone, the sound intensity, and the rhythm of the predetermined sound output by the sound-generating piece changes accordingly.

In one optional embodiment, the first control module 820 includes a first control unit and/or a second control unit. The first control unit is configured to control the motor to vibrate according to the oral care pressure change information and a first predetermined rule, to output at least one of the tone and the sound intensity of the predetermined sound to generate the corresponding change. The second control unit is configured to control the motor to vibrate to output the rhythm of the predetermined sound to generate the corresponding change according to the oral care motion change information and a second predetermined rule.

In one optional embodiment, the first control unit includes a first adjusting subunit and/or a second adjusting subunit. The first adjusting subunit is configured to adjust a vibration frequency of the motor according to the oral care pressure change information to control the motor to vibrate to output the tone of the predetermined sound to generate the corresponding change. The second adjusting subunit is configured to adjust a vibration amplitude of the motor according to the oral care pressure change information to control the motor to vibrate to output the sound intensity of the predetermined sound to generate the corresponding change.

In one optional embodiment, the oral care pressure change information includes a change direction and a change amount of an oral care pressure on a care piece of the oral care device.

The first adjusting subunit is specifically configured to determine a first adjustment direction corresponding to the vibration frequency of the motor according to the change direction of the oral care pressure, determine a first adjustment amount corresponding to the vibration frequency of the motor according to the change amount of the oral care pressure; and adjust the vibration frequency of the motor according to the first adjustment direction and the first adjustment amount.

The second adjusting subunit is specifically configured to determine a second adjustment direction corresponding to the vibration amplitude of the motor according to the change direction of the oral care pressure, determine a second adjustment amount corresponding to the vibration amplitude of the motor according to the change amount of the oral care pressure, and adjust the vibration amplitude of the motor according to the second adjustment direction and the second adjustment amount.

In one optional embodiment, when a changing direction of the oral care pressure is kept unchanged, the first adjustment direction is opposite to the second adjustment direction.

In one optional embodiment, the greater the oral care pressure of the oral care device, the lower the vibration frequency of the motor, so that the tone of the predetermined sound output by vibrations of the motor is lower; and/or the greater the oral care pressure, the greater the vibration amplitude of the motor, so that the sound intensity of the predetermined sound output by the vibrations of the motor is greater.

The smaller the oral care pressure, the higher the vibration frequency of the motor, so that the tone of the predetermined sound output by the vibrations of the motor is higher, and/or, the smaller the oral care pressure, the smaller the vibration amplitude of the motor, so that the sound intensity of the predetermined sound output by the vibrations of the motor is smaller.

In one optional embodiment, when the oral care pressure of the oral care device is not less than a first predetermined pressure value and is not greater than a second predetermined pressure value, the first control unit is specifically configured to control the motor to vibrate within a first frequency range and a first amplitude range according to the oral care pressure change information, so as to control the motor to vibrate to output at least one of the tone and the sound intensity of the predetermined sound to generate the corresponding change. The second predetermined pressure value is greater than the first predetermined pressure value.

In one optional embodiment, the control device 800 of the oral care device further includes a fifth control module and a sixth control module. When the oral care pressure is less than the first predetermined pressure value, the fifth control module is configured to control the motor to vibrate in a second frequency range and a second amplitude range or control the motor to vibrate to output a pressure-too-small prompt sound. A minimum value of the first frequency range is not less than a maximum value of the second frequency range, and a minimum value of the first amplitude range is not less than a maximum value of the second amplitude range.

When the oral care pressure is greater than the second predetermined pressure value, the sixth control module is configured to control the motor to vibrate at a threshold frequency and a threshold amplitude or control the motor to vibrate to output a pressure-excessive prompt sound. The threshold frequency is a boundary frequency within the first frequency range, and the threshold amplitude is a boundary amplitude within the first amplitude range.

In one optional embodiment, when the oral care pressure is switched from being not less than the first predetermined pressure value to being less than the first predetermined pressure value, and when a duration of the oral care pressure being less than the first predetermined pressure value is greater than a predetermined duration, the fifth control module is specifically configured to adjust the vibration frequency of the motor to the second frequency range, and reduce the vibration amplitude of the motor to the second amplitude range.

In one optional embodiment, the control device 800 of the oral care device further includes a seventh control module. When the oral care device is turned on, the seventh control module is configured to control the motor to vibrate in the second frequency range and the second amplitude range.

When it is detected that the oral care pressure is greater than the first predetermined pressure value, the first control unit controls the motor to vibrate in the first frequency range and the first amplitude range according to the oral care pressure change information, so as to control the motor to vibrate to output the tone and the sound intensity of the predetermined sound through to generate the corresponding change.

In one optional embodiment, the fifth control module unit includes a determining subunit and a controlling subunit. The determining subunit is configured to determine the change information of the oral care frequency of the oral care device according to the number of times of the oral care performed back and forth in the horizontal direction or the vertical direction within a unit time.

The control subunit is configured to control the motor to vibrate to output the rhythm of the predetermined sound to generate the corresponding change according to the change information of the oral care frequency.

In one optional embodiment, when the oral care frequency increases, the control subunit is specifically configured to control the motor to vibrate to output a faster rhythm of the predetermined sound. When the oral care frequency decreases, the control subunit is specifically configured to control the motor to vibrate to output a slower rhythm of the predetermined sound.

In one optional embodiment, when the oral care frequency changes or the frequency range of the oral care frequency changes, the rhythm of the predetermined sound output by the vibrations of the motor changes.

In one optional embodiment, when the oral care frequency is not less than a first predetermined frequency value and not greater than a second predetermined frequency value, the control subunit is specifically configured to control the motor to vibrate and output the rhythm of the predetermined sound to generate the corresponding change according to the change information of the oral care frequency. The second predetermined frequency value is greater than the first predetermined frequency value.

In one optional embodiment, the control device 800 of the oral care apparatus further includes an eighth control module and a ninth control module. When the oral care frequency is less than the first predetermined frequency value, the eighth control module is configured to control the motor to vibrate to output the predetermined sound at a first rhythm. When the oral care frequency is greater than the second predetermined frequency value, the ninth control module is configured to control the motor to vibrate to output the predetermined sound at a second rhythm. The first rhythm is less than the second rhythm.

In one optional embodiment, when the oral care pressure changes from 0 to greater than the first predetermined pressure value, the control subunit is configured to control the motor to vibrate to output the rhythm of the predetermined sound to generate the corresponding change according to the change information of the oral care frequency.

In one optional embodiment, the first control module 820 includes a third control unit and a fourth control unit.

When a pure music enjoyment mode of the oral care device is turned on, the third control unit is configured to control the motor to vibrate to output at least one of the tone and the sound intensity of the predetermined sound to generate the corresponding change based on the first predetermined rule according to the oral care pressure change information. When a variation mode of the oral care device is turned on, the fourth control unit is configured to control the motor to vibrate to output the rhythm of the predetermined sound to generate the corresponding change based on the second predetermined rule according to the oral care motion change information.

In one optional embodiment, the control device 800 of the oral care apparatus further includes a tenth control module and an eleventh control module.

When the oral care pressure of the oral care device is not less than the first predetermined pressure value and is not greater than the second predetermined pressure value, the tenth control module is configured to control the motor to vibrate at a target vibration frequency and a target vibration amplitude to output the predetermined sound at a target tone and a target sound intensity.

When the oral care pressure is less than the first predetermined pressure value or greater than the second predetermined pressure value, the eleventh control module is configured to control at least one of the tone, the sound intensity, and the rhythm of the predetermined sound output by the vibrations of the motor to generate the corresponding change. When the oral care pressure is less than the first predetermined pressure value or greater than the second predetermined pressure value, the tone of the predetermined sound output by the vibrations of the motor is less than the target tone, and/or the sound intensity of the predetermined sound output by the vibrations of the motor is less than the target sound intensity, and/or the rhythm of the predetermined sound output by the vibrations of the motor is less than a corresponding rhythm when the oral care pressure of the oral care device is not less than the first predetermined pressure value and not greater than the second predetermined pressure value.

A division of the modules in the control device of the oral care device is only for illustration. In other embodiments, the control device of the oral care device may be divided into different modules as needed to complete all or part of the functions of the control device of the oral care device. Each of the modules of the control device of the oral care device of the embodiments of the present disclosure may be a computer program. The computer program may be run on a terminal device, a server, or the oral care device. Program modules constituted by the computer program may be stored in a memory of the terminal device, the server, or the oral care device. When the computer program is executed by the processor, all or part of the steps of the control method of the oral care device described in the embodiments of the present disclosure are implemented.

Please refer to FIG. 9, FIG. 9 is another structural schematic diagram of the oral care device according to one optional embodiment of the present disclosure. As shown in FIG. 9, the oral care device 900 includes at least one processor 910, a pressure detection module 920, a user interface 930, a memory 940, at least one network interface 950, a sound-generating piece 960, and at least one communication bus 970.

The oral care device 900 may be, but is not limited to, the oral cleaning device such as an electric toothbrush.

The at least one communication bus 960 is configured to realize connection and communication between the various components of the oral care device 900.

The detection element 920 includes, but not limited to, a pressure detection element, an oral care motion detection element, etc. The pressure detection element may be, but is not limited to, detect a pressure on a care piece of the oral care device through any pressure sensing method such as through a strain gauge, a Hall element magnetic induction, a capacitor, a resistor, an inductor, etc. The pressure detection element may be disposed at a motor shaft of the oral care device, the care piece (such as a brush head), a housing of a handle (such as a brush handle), a movement, a motor shell, or other position of the oral care device, which is not limited thereto. The detection element may, but is not limited to, detect the oral care frequency of the oral care device performing the oral care back and forth through any sensor such as an acceleration sensor or a multi-axis sensor. For instance, the oral care frequency includes, but is not limited to, an upper-lower oral care frequency (i.e., a upper-lower vertical brushing frequency) and left-right oral care frequency (i.e., a left-right horizontal brushing frequency).

The sound-generating piece 960 may be, but is not limited to, a motor or a speaker. When the sound-generating piece 960 is the motor, the care piece of the oral care device generates a certain swing amplitude through vibrations of the motor, thereby breaking down toothpaste pasted on the care pieced into fine foam to achieve deep cleaning of the teeth. Further, the motor vibrates to drive the care piece to output a predetermined sound in a bone conduction manner. When the sound-generating piece 960 is the speaker, the oral care device directly emit the predetermined sound through the speaker, which is not limited thereto.

The user interface 930 includes a display screen and a camera. Optionally, the user interface 930 may further includes a standard wired interface and a wireless interface.

Optionally, the at least one network interface 950 includes a BLUETOOTH module, a near field communication (NFC) module, a wireless fidelity (Wi-Fi) module, etc.

The at least one processor 910 includes one or more processing cores. The at least one processor 910 uses various interfaces and lines to connect various parts of the oral care device 900. The processor 910 executes various functions and processes data of the oral care device 900 by running or executing instructions, programs, code sets or instruction sets stored in the memory 940, and calling data stored in the memory 940. Optionally, the at least one processor 910 is implemented by at least one hardware of a digital signal processing (DSP), a field programmable gate array (FPGA), and a programmable logic array (PLA). The at least one processor 910 may integrate one or a combination of a central processing unit (CPU), a graphics processing unit (GPU), and a modem. The CPU mainly processes an operating system and an application for the oral care device 900. The GPU is responsible for rendering and drawing content to be displayed on the display screen. The modem is configured to process wireless communications. It is understood that the modem may not be integrated into the at least one processor 910, but implemented by a chip.

The memory 940 may be a random access memory (RAM) or a read-only memory (ROM). Optionally, the memory 940 may be a non-transitory computer-readable medium. The memory 940 is configured to store the instructions, the programs, the codes, the code sets or the instruction sets. The memory 940 includes a program storage area and a data storage area. The program storage area stores the instructions for implementing the operating system, the instructions for at least one function (such as a receiving function, a control function, a determination function, etc.), the instructions for implementing the controls method of the present disclosure, etc. The data storage area stores data involved in the control method of the present disclosure, etc. Optionally, the memory 940 may be at least one storage device located away from the at least one processor 910. As shown in FIG. 9, the memory 940 served as a computer storage medium may include the operating system, a network communication module, a user interface module, and program instructions.

In some optional embodiments, the oral care device 900 may be the control device of the oral care device mentioned above, or may be the electric toothbrush, etc., which is not limited thereto. In the oral care device shown in FIG. 9, the at least one processor 910 is configured to call program instructions stored in the memory 940 and execute following steps of acquiring at least one of oral care motion change information and oral care pressure change information of the oral care device; and controlling, according to at least one of the oral care motion change information and the oral care pressure change information, the oral care device to output at least one of a tone, a sound intensity, and a rhythm of a predetermined sound to generate a corresponding change.

In some optional embodiments, when the at least one processor 910 of executes the steps of controlling, according to at least one of the oral care motion change information and the oral care pressure change information, the oral care device to output at least one of the tone, the sound intensity, and the rhythm of the predetermined sound to generate the corresponding change, the at least one processor 910 is specifically configured to control the sound-generating piece to output at least one of the tone, the sound intensity, and the rhythm of the predetermined sound to generate the corresponding change according to at least one of the oral care motion change information and the oral care pressure change information.

In some optional embodiments, the sound-generating piece includes at least one of the speaker and the motor.

In some optional embodiments, when the at least one processor 910 execute the step of controlling, according to at least one of the oral care motion change information and the oral care pressure change information, the sound-generating piece to output at least one of the tone, the sound intensity, and the rhythm of the predetermined sound to generate the corresponding change, the at least one processor 910 is specifically configured to control the motor to vibrate to output at least one of the tone, the sound intensity, and the rhythm of the predetermined sound to generate the corresponding change according to at least one of the oral care motion change information and the oral care pressure change information.

In some optional embodiments, when the at least one processor 910 execute the step of controlling, according to at least one of the oral care motion change information and the oral care pressure change information, the sound-generating piece to output at least one of the tone, the sound intensity, and the rhythm of the predetermined sound to generate the corresponding change, the at least one processor 910 is specifically configured to control the speaker in a predetermined manner to output at least one of the tone, the sound intensity, and the rhythm of the predetermined sound to generate the corresponding change according to at least one of the oral care motion change information and the oral care pressure change information.

In some optional embodiments, before the step of controlling, according to at least one of the oral care motion change information and the oral care pressure change information, the sound-generating piece to output at least one of the tone, the sound intensity, and the rhythm of the predetermined sound to generate the corresponding change, the at least one processor 910 is configured to acquire the predetermined sound and generate a first driving signal according to the predetermined sound.

When the at least one processor 910 execute the step of controlling, according to at least one of the oral care motion change information and the oral care pressure change information, the sound-generating piece to output at least one of the tone, the sound intensity, and the rhythm of the predetermined sound to generate the corresponding change, the at least one processor 910 is specifically configured to update the first driving signal according to at least one of the oral care motion change information and the oral care pressure change information to obtain a second driving signal; and drive the sound-generating piece to output at least one of the tone, the sound intensity, and the rhythm of the predetermined sound to generate the corresponding change according to the second driving signal.

In some optional embodiments, the sound-generating piece includes the speaker, and the oral care device further includes a power amplifier. When the at least one processor 910 drives the sound-generating piece to output at least one of the tone, the sound intensity, and the rhythm of the predetermined sound to generate the corresponding change according to the second driving signal, the at least one processor 910 is specifically configured to transmit the second driving signal to the power amplifier to make the power amplifier to amplify the second driving signal, and drive the speaker to output at least one of the tone, the sound intensity, and the rhythm of the predetermined sound to generate the corresponding change according to an amplified second driving signal.

In some optional embodiments, the at least one processor 910 is configured to receive a sound intensity change instruction and control a driving voltage corresponding to the sound-generating piece to change in response to the sound intensity change instruction, so that the sound-generating piece outputs the sound intensity of the predetermined sound to generate the corresponding change.

In some optional embodiments, the at least one processor 910 is configured to acquire current oral care parameters of the oral care device and determine whether the current oral care parameters are within a predetermined parameter range. The at least one processor 910 is further configured to control the oral care device to output at least one of the tone, the sound intensity, and the rhythm of the predetermined sound to generate the corresponding change when the current oral care parameters are outside the predetermined parameter range.

In some optional embodiments, after executing the step of determining whether the current oral care parameters are within the predetermined parameter range, the at least one processor 910 is configured to control the oral care device to output the tone, the sound intensity, and the rhythm of the predetermined sound being unchanged when the current oral care parameters are within the predetermined parameter range. Alternatively, the at least one processor 910 is configured to control the oral care device to output the tone, the sound intensity, and the rhythm of the predetermined sound being unchanged when the current oral care parameters are within a target parameter interval. The at least one processor 910 is further configured to control the oral care device to output at least one of the tone, the sound intensity, and the rhythm of the predetermined sound to generate the corresponding change according to a predetermined rule when the current oral care parameters are within the predetermined parameter range and outside the target parameter interval.

In some optional embodiments, the current oral care parameters include a current oral care pressure, and the predetermined parameter range includes a predetermined pressure range; and/or the predetermined rule includes that at least one of the tone and the sound intensity reduces first and then increases, at least one of the tone and the sound intensity increases first and then reduces, at least one of the tone and the sound intensity increases at a predetermined amplitude, at least one of the tone and the sound intensity decreases with a predetermined amplitude, or at least one of the tone and the sound intensity repeatedly changes within a predetermined range; and/or the rhythm speeds up first and then slows down, or the rhythm slows down first then speeds up, or the rhythm slows down at the predetermined amplitude, or the rhythm speeds up at the predetermined amplitude, or the rhythm repeatedly changes within the predetermined range.

In some optional embodiments, the oral care device is communicated with an electronic device, and the electronic device includes a loudspeaker. The at least one processor 910 is further configured to acquire current oral care parameters of the oral care device. The sending module is configured to transmit the current oral care parameters to the electronic device. When the current oral care parameters are outside the predetermined parameter rang, the at least one processor 910 is configured to control the loudspeaker to output at least one of the tone, the sound intensity, and the rhythm of the predetermined sound.

In some optional embodiments, the electronic device includes at least one of a central console, the loudspeaker, and a terminal.

In some optional embodiments, the at least one processor 910 is configured to acquire the current oral care parameters, and is configured to control the sound-generating piece to output at least one of the tone, the sound intensity, and the rhythm of the predetermined sound to generate the corresponding change according to the change information of the parameter range of the current oral care parameters, Specifically, when the parameter range of the current oral care parameters changes, at least one of the tone, the sound intensity, and the rhythm of the predetermined sound output by the sound-generating piece changes accordingly.

In some optional embodiments, when the at least one processor 910 executes the steps of controlling, according to at least one of the oral care motion change information and the oral care pressure change information, the motor to vibrate to output at least one of the tone, the sound intensity, and the rhythm of the predetermined sound to generate the corresponding change, the at least one processor 910 is specifically configured to control the motor to vibrate according to the oral care pressure change information and a first predetermined rule, to output at least one of the tone and the sound intensity of the predetermined sound to generate the corresponding change, and/or the at least one processor 910 is configured to control the motor to vibrate to output the rhythm of the predetermined sound to generate the corresponding change according to the oral care motion change information and a second predetermined rule.

In some optional embodiments, when executing the step of controlling the motor to vibrate according to the oral care pressure change information and the first predetermined rule, to output at least one of the tone and the sound intensity of the predetermined sound to change, the at least one processor 910 is configured to adjust a vibration frequency of the motor according to the oral care pressure change information to control the motor to vibrate to output the tone of the predetermined sound to generate the corresponding change, and/or the at least one processor 910 is configured to adjust a vibration amplitude of the motor according to the oral care pressure change information to control the motor to vibrate to output the sound intensity of the predetermined sound to generate the corresponding change.

In some optional embodiments, the oral care pressure change information includes a change direction and a change amount of an oral care pressure on a care piece of the oral care device. When the at least one processor 910 adjusts the vibration frequency of the motor according to the oral care pressure change information, the at least one processor 910 is specifically configured to determine a first adjustment direction corresponding to the vibration frequency of the motor according to the change direction of the oral care pressure, determine a first adjustment amount corresponding to the vibration frequency of the motor according to the change amount of the oral care pressure; and adjust the vibration frequency of the motor according to the first adjustment direction and the first adjustment amount.

When the at least one processor 910 adjusts the vibration amplitude of the motor according to the oral care pressure change information, the at least one processor 910 is specifically configured to determine a second adjustment direction corresponding to the vibration amplitude of the motor according to the change direction of the oral care pressure, determine a second adjustment amount corresponding to the vibration amplitude of the motor according to the change amount of the oral care pressure, and adjust the vibration amplitude of the motor according to the second adjustment direction and the second adjustment amount.

In some optional embodiments, when a changing direction of the oral care pressure is kept unchanged, the first adjustment direction is opposite to the second adjustment direction.

In some optional embodiments, the greater the oral care pressure of the oral care device, the lower the vibration frequency of the motor, so that the tone of the predetermined sound output by vibrations of the motor is lower; and/or the greater the oral care pressure, the greater the vibration amplitude of the motor, so that the sound intensity of the predetermined sound output by the vibrations of the motor is greater.

The smaller the oral care pressure, the higher the vibration frequency of the motor, so that the tone of the predetermined sound output by the vibrations of the motor is higher, and/or, the smaller the oral care pressure, the smaller the vibration amplitude of the motor, so that the sound intensity of the predetermined sound output by the vibrations of the motor is smaller.

In some optional embodiments, when executing the step of controlling the motor to vibrate according to the oral care pressure change information and the first predetermined rule, to output at least one of the tone and the sound intensity of the predetermined sound to change, the at least one processor 910 is specifically configured to control the motor to vibrate within a first frequency range and a first amplitude range according to the oral care pressure change information when the oral care pressure of the oral care device is not less than a first predetermined pressure value and is not greater than a second predetermined pressure value, so as to control the motor to vibrate to output at least one of the tone and the sound intensity of the predetermined sound to generate the corresponding change. The second predetermined pressure value is greater than the first predetermined pressure value.

In some optional embodiments, when the oral care pressure is less than the first predetermined pressure value, the at least one processor 910 is configured to control the motor to vibrate in a second frequency range and a second amplitude range or control the motor to vibrate to output a pressure-too-small prompt sound. A minimum value of the first frequency range is not less than a maximum value of the second frequency range, and a minimum value of the first amplitude range is not less than a maximum value of the second amplitude range. When the oral care pressure is greater than the second predetermined pressure value, the at least one processor 910 is configured to control the motor to vibrate at a threshold frequency and a threshold amplitude or control the motor to vibrate to output a pressure-excessive prompt sound. The threshold frequency is a boundary frequency within the first frequency range, and the threshold amplitude is a boundary amplitude within the first amplitude range.

In some optional embodiments, when the at least one processor 910 executes the step of controlling the motor to vibrate in the second frequency range and the second amplitude range when the oral care pressure is less than the first predetermined pressure value, when the oral care pressure is switched from being not less than the first predetermined pressure value to being less than the first predetermined pressure value, and when a duration of the oral care pressure being less than the first predetermined pressure value is greater than a predetermined duration, the at least one processor 910 is specifically configured to adjust the vibration frequency of the motor to the second frequency range, and reduce the vibration amplitude of the motor to the second amplitude range.

In some optional embodiments, before the at least one processor 910 acquires the oral care motion change information and/or the oral care pressure change information of the oral care device, the at least one processor 910 is configured to control the motor to vibrate in the second frequency range and the second amplitude range when the oral care device is turned on.

When executing the step of controlling the motor to vibrate according to the oral care pressure change information and the first predetermined rule, to output at least one of the tone and the sound intensity of the predetermined sound to change, the at least one processor 910 is specifically configured to control the motor to vibrate in the first frequency range and the first amplitude range according to the oral care pressure change information, so as to control the motor to vibrate to output the tone and the sound intensity of the predetermined sound through to generate the corresponding change, when it is detected that the oral care pressure is greater than the first predetermined pressure value.

In some optional embodiments, when executing the step of controlling the motor to vibrate to output the rhythm of the predetermined sound to generate the corresponding change according to the oral care motion change information and the second predetermined rule the at least one processor 910 is specifically configured to determine the change information of the oral care frequency of the oral care device. The oral care frequency represents the number of times of the oral care performed back and forth in the horizontal direction or the vertical direction within a unit time by the oral care device. The at least one processor 910 is configured to control the motor to vibrate to output the rhythm of the predetermined sound to generate the corresponding change according to the change information of the oral care frequency.

In some optional embodiments, when the at least one processor 910 controls the motor to vibrate to output the rhythm of the predetermined sound to generate the corresponding change according to the change information of the oral care frequency, the at least one processor 910 is specifically configured to control the motor to vibrate to output a faster rhythm of the predetermined sound. When the oral care frequency decreases, the control subunit is specifically configured to control the motor to vibrate to output a slower rhythm of the predetermined sound, when the oral care frequency increases.

In some optional embodiments, when the oral care frequency changes or the frequency range of the oral care frequency changes, the rhythm of the predetermined sound output by the vibrations of the motor changes.

In some optional embodiments, when the at least one processor 910 controls the motor to vibrate to output the rhythm of the predetermined sound to generate the corresponding change according to the change information of the oral care frequency, the at least one processor 910 is specifically configured to control the motor to vibrate and output the rhythm of the predetermined sound to generate the corresponding change according to the change information of the oral care frequency, when the oral care frequency is not less than a first predetermined frequency value and not greater than a second predetermined frequency value. The second predetermined frequency value is greater than the first predetermined frequency value.

In some optional embodiments, when the oral care frequency is less than the first predetermined frequency value, the at least one processor 910 is configured to control the motor to vibrate to output the predetermined sound at a first rhythm. When the oral care frequency is greater than the second predetermined frequency value, the at least one processor 910 is configured to control the motor to vibrate to output the predetermined sound at a second rhythm. The first rhythm is less than the second rhythm.

In some optional embodiments, when the at least one processor 910 controls the motor to vibrate and output the rhythm of the predetermined sound to generate the corresponding change according to the change information of the oral care frequency, and when the oral care pressure changes from 0 to greater than the first predetermined pressure value, the at least one processor 910 is configured to control the motor to vibrate to output the rhythm of the predetermined sound to generate the corresponding change according to the change information of the oral care frequency.

In some optional embodiments, the oral care device has a pure music enjoyment mode and a variation mode. When the at least one processor 910 executes the step of controlling, according to at least one of the oral care motion change information and the oral care pressure change information, the motor to output at least one of the tone, the sound intensity, and the rhythm of the predetermined sound to generate the corresponding change, the at least one processor 910 is specifically configured to control the motor to vibrate to output at least one of the tone and the sound intensity of the predetermined sound to generate the corresponding change based on the first predetermined rule according to the oral care pressure change information. When the pure music enjoyment mode of the oral care device is turned on. Further, when the variation mode of the oral care device is turned on, the at least one processor 910 is configured to control the motor to vibrate to output the rhythm of the predetermined sound to generate the corresponding change based on the second predetermined rule according to the oral care motion change information.

In some optional embodiments, when the oral care pressure of the oral care device is not less than the first predetermined pressure value and is not greater than the second predetermined pressure value, the at least one processor 910 is configured to control the motor to vibrate at a target vibration frequency and a target vibration amplitude to output the predetermined sound at a target tone and a target sound intensity.

When the oral care pressure is less than the first predetermined pressure value or greater than the second predetermined pressure value, the at least one processor 910 is configured to control at least one of the tone, the sound intensity, and the rhythm of the predetermined sound output by the vibrations of the motor to generate the corresponding change. When the oral care pressure is less than the first predetermined pressure value or greater than the second predetermined pressure value, the tone of the predetermined sound output by the vibrations of the motor is less than the target tone, and/or the sound intensity of the predetermined sound output by the vibrations of the motor is less than the target sound intensity, and/or the rhythm of the predetermined sound output by the vibrations of the motor is less than a corresponding rhythm when the oral care pressure of the oral care device is not less than the first predetermined pressure value and not greater than the second predetermined pressure value.

The embodiments of the present disclosure further provide a computer storage medium storing instructions therein. When the instructions are executed on a computer or at least one processor, the computer or the at least one processor executes one or more steps in any of the embodiments mentioned above. When various component modules of the control device of the oral care device are implemented in the form of software functional units and sold or used as an independent product, the various component modules may be stored in the computer storage medium.

Those skilled in the art can understand that all or part of the processes in the above-mentioned embodiments can be implemented by instructing the relevant hardware through the computer programs, and the computer programs may be stored in the computer-readable storage medium. When the computer programs are executed, the processes of the embodiments of the present disclosure are executed. The storage medium includes the ROM, the RAM, a magnetic disk, an optical disk, or other media that are able to store the program codes. In the absence of conflict, the technical features in the embodiments and implementation schemes of the present disclosure can be combined arbitrarily.

The embodiments described above are merely optional embodiments of the present disclosure and are not intended to limit the scope of the present disclosure. Without departing from the design spirit of the present disclosure, various modifications and improvements made to the technical solutions of the present disclosure by those skilled in the art should fall within the protection scope determined by the claims of the present disclosure.

What is claimed is:

1. A control method of an oral care device, comprising steps:
acquiring at least one of oral care motion change information and oral care pressure change information of the oral care device; and
controlling, according to at least one of the oral care motion change information and the oral care pressure change information, the oral care device to output at least one of a tone, a sound intensity, and a rhythm of a predetermined sound to generate a corresponding change.

2. The control method according to claim 1, wherein the oral care device comprises a sound-generating piece, the sound-generating piece comprises at least one of a speaker and a motor, and the steps of controlling, according to at least one of the oral care motion change information and the oral care pressure change information, the oral care device to output at least one of the tone, the sound intensity, and the rhythm of the predetermined sound to generate the corresponding change comprises a step:
controlling, according to at least one of the oral care motion change information and the oral care pressure change information, the sound-generating piece to output at least one of the tone, the sound intensity, and the rhythm of the predetermined sound to generate the corresponding change.

3. The control method according to claim 2, wherein the step of controlling, according to at least one of the oral care motion change information and the oral care pressure change information, the sound-generating piece to output at least one of the tone, the sound intensity, and the rhythm of the predetermined sound to generate the corresponding change comprises steps:
controlling, according to at least one of the oral care motion change information and the oral care pressure change information, the motor to vibrate to output at least one of the tone, the sound intensity, and the rhythm of the predetermined sound to generate the corresponding change; and/or
controlling, according to at least one of the oral care motion change information and the oral care pressure change information, the speaker in a predetermined manner to output at least one of the tone, the sound intensity, and the rhythm of the predetermined sound to generate the corresponding change.

4. The control method according to claim 2, wherein before the step of controlling, according to at least one of the oral care motion change information and the oral care pressure change information, the sound-generating piece to output at least one of the tone, the sound intensity, and the rhythm of the predetermined sound to generate the corresponding change, the control method further comprises steps:
acquiring the predetermined sound; and
generating a first driving signal according to the predetermined sound;
wherein the step of controlling, according to at least one of the oral care motion change information and the oral care pressure change information, the sound-generating piece to output at least one of the tone, the sound intensity, and the rhythm of the predetermined sound to generate the corresponding change comprises steps:
updating the first driving signal according to at least one of the oral care motion change information and the oral care pressure change information to obtain a second driving signal; and
driving, according to the second driving signal, the sound-generating piece to output at least one of the tone, the sound intensity, and the rhythm of the predetermined sound to generate the corresponding change.

5. The control method according to claim 4, wherein the sound-generating piece comprises the speaker, and the oral care device further comprises a power amplifier;
wherein the step of driving, according to the second driving signal, the sound-generating piece to output at least one of the tone, the sound intensity, and the rhythm of the predetermined sound to generate the corresponding change comprises:
transmitting the second driving signal to the power amplifier to make the power amplifier to amplify the second driving signal, and driving the speaker to output at least one of the tone, the sound intensity, and the rhythm of the predetermined sound to generate the corresponding change according to an amplified second driving signal.

6. The control method according to claim 2, wherein the control method further comprises steps:
receiving a sound intensity change instruction; and
in response to the sound intensity change instruction, controlling a driving voltage corresponding to the sound-generating piece to change, so that the sound-generating piece outputs the sound intensity of the predetermined sound to generate the corresponding change.

7. The control method according to claim 1, wherein the control method further comprises steps:
acquiring current oral care parameters of the oral care device;
determining whether the current oral care parameters are within a predetermined parameter range; and
when the current oral care parameters are outside the predetermined parameter range, controlling the oral care device to output at least one of the tone, the sound intensity, and the rhythm of the predetermined sound to generate the corresponding change.

8. The control method according to claim 7, wherein after the step of determining whether the current oral care parameters are within the predetermined parameter range, the control method further comprises steps:
when the current oral care parameters are within the predetermined parameter range, controlling the oral care device to output the tone, the sound intensity, and the rhythm of the predetermined sound being unchanged;
when the current oral care parameters are within a target parameter interval, controlling the oral care device to output the tone, the sound intensity, and the rhythm of the predetermined sound being unchanged; or
when the current oral care parameters are within the predetermined parameter range and outside the target parameter interval, controlling the oral care device to output at least one of the tone, the sound intensity, and the rhythm of the predetermined sound to generate the corresponding change according to a predetermined rule.

9. The control method according to claim 7, wherein the current oral care parameters comprise a current oral care pressure, and the predetermined parameter range comprises a predetermined pressure range; and/or
the predetermined rule comprises that at least one of the tone and the sound intensity reduces first and then increases, at least one of the tone and the sound intensity increases first and then reduces, at least one of the tone and the sound intensity increases at a predetermined amplitude, at least one of the tone and the sound intensity decreases with a predetermined amplitude, or at least one of the tone and the sound intensity repeatedly changes within a predetermined range; and/or
the rhythm speeds up first and then slows down, the rhythm slows down first then speeds up, the rhythm slows down at the predetermined amplitude, the rhythm speeds up at the predetermined amplitude, or the rhythm repeatedly changes within the predetermined range.

10. The control method according to claim 2, wherein the oral care device is communicated with an electronic device, the electronic device comprises a loudspeaker, and the control method further comprises steps:
acquiring current oral care parameters of the oral care device; and
transmitting the current oral care parameters to the electronic device, and when the current oral care parameters are outside the predetermined parameter range, controlling, by the electronic device, the loudspeaker to output at least one of the tone, the sound intensity, and the rhythm of the predetermined sound to generate the corresponding change.

11. The control method according to claim 3, wherein the step of controlling, according to at least one of the oral care motion change information and the oral care pressure change information, the motor to vibrate to output at least one of the tone, the sound intensity, and the rhythm of the predetermined sound to generate the corresponding change comprises steps:
controlling, according to the oral care pressure change information, the motor to vibrate, based on a first predetermined rule, to output at least one of the tone and the sound intensity of the predetermined sound to generate the corresponding change; and/or
controlling, according to the oral care motion change information, the motor to vibrate, based on a second predetermined rule, to output the rhythm of the predetermined sound to generate the corresponding change.

12. The control method according to claim 11, wherein the step of controlling, according to the oral care pressure change information, the motor to vibrate, based on the first predetermined rule, to output at least one of the tone and the sound intensity of the predetermined sound to generate the corresponding change comprises steps:
adjusting a vibration frequency of the motor according to the oral care pressure change information to control the motor to vibrate to output the tone of the predetermined sound to generate the corresponding change; and/or
adjusting a vibration amplitude of the motor according to the oral care pressure change information to control the motor to vibrate to output the sound intensity of the predetermined sound to generate the corresponding change.

13. The control method according to claim 12, wherein the oral care pressure change information comprises a change direction and a change amount of an oral care pressure on a care piece of the oral care device;
wherein adjusting the vibration frequency of the motor according to the oral care pressure change information comprises steps:
determining a first adjustment direction corresponding to the vibration frequency of the motor according to the change direction of the oral care pressure;
determining a first adjustment amount corresponding to the vibration frequency of the motor according to the change amount of the oral care pressure; and
adjusting the vibration frequency of the motor according to the first adjustment direction and the first adjustment amount;
wherein adjusting the vibration amplitude of the motor according to the oral care pressure change information comprises:
determining a second adjustment direction corresponding to the vibration amplitude of the motor according to the change direction of the oral care pressure;
determining a second adjustment amount corresponding to the vibration amplitude of the motor according to the change amount of the oral care pressure; and
adjusting the vibration amplitude of the motor according to the second adjustment direction and the second adjustment amount.

14. The control method according to claim 13, wherein when the changing direction of the oral care pressure is kept unchanged, the first adjustment direction is opposite to the second adjustment direction.

15. The control method according to claim 11, wherein the greater the oral care pressure of the oral care device, the lower the vibration frequency of the motor, so that the tone of the predetermined sound output by vibrations of the motor is lower; and/or the greater the oral care pressure, the greater the vibration amplitude of the motor, so that the sound intensity of the predetermined sound output by the vibrations of the motor is greater;
the smaller the oral care pressure, the higher the vibration frequency of the motor, so that the tone of the predetermined sound output by the vibrations of the motor is higher; and/or, the smaller the oral care pressure, the smaller the vibration amplitude of the motor, so that the sound intensity of the predetermined sound output by the vibrations of the motor is smaller.

16. The control method according to claim 11, wherein the step of controlling, according to the oral care pressure change information, the motor to vibrate, based on the first predetermined rule, to output at least one of the tone and the sound intensity of the predetermined sound to generate the corresponding change comprises a step:
when the oral care pressure of the oral care device is not less than a first predetermined pressure value and is not greater than a second predetermined pressure value, controlling the motor to vibrate within a first frequency range and a first amplitude range according to the oral care pressure change information, so as to control the motor to vibrate to output at least one of the tone and the sound intensity of the predetermined sound to generate the corresponding change;
wherein the second predetermined pressure value is greater than the first predetermined pressure value.

17. The control method according to claim 16, wherein the control method further comprises steps:
when the oral care pressure is less than the first predetermined pressure value, controlling the motor to vibrate in a second frequency range and a second amplitude range or controlling the motor to vibrate to output a pressure-too-small prompt sound; wherein a minimum value of the first frequency range is not less than a maximum value of the second frequency range, and a minimum value of the first amplitude range is not less than a maximum value of the second amplitude range; and
when the oral care pressure is greater than the second predetermined pressure value, controlling the motor to vibrate at a threshold frequency and a threshold amplitude or controlling the motor to vibrate to output a pressure-excessive prompt sound, wherein the threshold frequency is a boundary frequency within the first frequency range, and the threshold amplitude is a boundary amplitude within the first amplitude range.

18. The control method according to claim 17, wherein the step of controlling the motor to vibrate in the second frequency range and the second amplitude range when the oral care pressure is less than the first predetermined pressure value comprises steps:
when the oral care pressure is switched from being not less than the first predetermined pressure value to being less than the first predetermined pressure value, and when a duration of the oral care pressure being less than the first predetermined pressure value is greater than a predetermined duration, adjusting the vibration frequency of the motor to the second frequency range, and reducing the vibration amplitude of the motor to the second amplitude range.

19. A control device of an oral care device, comprising:
an acquisition module; and
a first control module;
wherein the acquisition module is configured to acquire at least one of oral care motion change information and oral care pressure change information of the oral care device;
wherein the first control module is configured to control the oral care device to output at least one of a tone, a sound intensity, and a rhythm of a predetermined sound to generate a corresponding change according to at least one of the oral care motion change information and the oral care pressure change information.

20. An oral care device, comprising:
a memory; and
a processor;
wherein the memory is connected to the processor, the memory is configured to store executable program codes; the processor runs programs corresponding to the executable program codes by reading the executable program codes stored in the memory, and the processor runs the programs to execute steps:
acquiring at least one of oral care motion change information and oral care pressure change information of the oral care device; and
controlling, according to at least one of the oral care motion change information and the oral care pressure change information, the oral care device to output at least one of a tone, a sound intensity, and a rhythm of a predetermined sound to generate a corresponding change.
